(19) United States
(12) Patent Application Publication
(10) Pub. No.: US 2025/0155397 A1
(43) Pub. Date: May 15, 2025

(54) IDENTIFICATION OF CLAYS IN POROUS MEDIA BY INTEGRATING ELECTROMAGNETIC MEASUREMENTS AND TEMPERATURE GRADIENT ANALYSIS

(71) Applicants: Schlumberger Technology Corporation, Sugar Land, TX (US); Saudi Arabian Oil Company, Dhahran (SA)

(72) Inventors: Mohammed Fadhel Al-Hamad, Dhahran (SA); Wael Abdallah, Sugar Land, TX (US); Ahmad Mohamad AlZoukani, Dhahran (SA); Shouxiang Ma, Dhahran (SA); Mustafa Huwaider, Dhahran (SA)

(21) Appl. No.: 18/508,376

(22) Filed: Nov. 14, 2023

(51) Int. Cl. G01N 27/14; E21B 49/00; E21B 49/02; G01N 1/44; G01N 33/24; G01V 3/20

(52) U.S. Cl. CPC G01N 27/14; E21B 49/005; E21B 49/02; G01N 1/44; G01N 33/24; G01V 3/20

(57) ABSTRACT

The present disclosure generally relates to systems and methods for determining clay content of porous media based on electromagnetic measurements and temperature gradient analysis. For example, in certain embodiments, a method includes sampling porous media of a reservoir formation; measuring a first resistivity value of the porous media at a first temperature; heating the porous media to a second temperature using a heating source; measuring a second resistivity value of the porous media at the second temperature; and determining whether the porous media contains clay based at least in part on the first and second resistivity values.

### BACKGROUND

This disclosure relates to systems and methods for determining clay content of porous media based on electromagnetic measurements and temperature gradient analysis.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as an admission of any kind.

Proper assessment of a hydrocarbon reservoir requires accurate petrophysical evaluation. For example, any misinterpretation of porosity and saturation can have a larger impact on reservoir productivity and overall recovery. The presence of clay minerals can affect the evaluation of shaly reservoirs and the determination of true reservoir saturation. Extensive studies have been conducted, investigating the effect of clays on resistivity log response. It has been found that the cation-exchange capacity (CEC) values directly correlate with the clay type and content.

Clay content identification is done using several commonly published methods, such as x-ray diffraction (XRD) and infrared (IR) spectroscopy analysis, Fourier transform infrared (FTIR) spectroscopy, thermal analysis, cation exchange capacity, and so forth, where measurements can be obtained on bulk core or fractions of clay. However, with all the available established methods, quantifying clay content can be relatively difficult. This difficulty is mainly due to the relative complexity of the clay minerals, governed by their unique structure and the variable chemical composition. Downhole clay identification is possible using high definition spectroscopy, nonetheless, it is not always possible to obtain such logs for every well.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

One embodiment of the present disclosure relates to a method that includes sampling porous media of a reservoir formation. The method also includes measuring a first resistivity value of the porous media at a first temperature. The method further includes heating the porous media to a second temperature using a heating source. In addition, the method includes measuring a second resistivity value of the porous media at the second temperature. The method also includes determining whether the porous media contains clay based at least in part on the first and second resistivity values.

Another embodiment of the present disclosure relates to a method that includes generating a conductivity enhancement ratio curve based at least in part on a plurality of resistivity values for a porous media sample at a plurality of temperatures. The method also includes determining a clay content of the porous media sample based at least in part on the conductivity enhancement ratio curve.

Another embodiment of the present disclosure relates to a system that includes a downhole well tool and a data processing system. The downhole well tool includes a heating source configured to heat a reservoir formation surrounding a wellbore while the downhole well tool is disposed within the wellbore. The downhole well tool also includes one or more sensors configured to detect resistivity values of the reservoir formation at a plurality of temperatures while the downhole well tool is disposed within the wellbore. The downhole well tool further includes communication circuitry configured to transmit the resistivity values of the reservoir formation. The data processing system is configured to receive the resistivity values of the reservoir formation from the downhole well tool. The data processing system is also configured to generate a conductivity enhancement ratio curve based at least in part the resistivity values of the reservoir formation. The data processing system is further configured to determine a clay content of the porous media sample based at least in part on the conductivity enhancement ratio curve.

Various refinements of the features noted above may be undertaken in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 is a schematic diagram of a well logging system that may obtain electromagnetic (EM) measurements that may be used to identify formation horizontal resistivity, vertical resistivity, dip and azimuth, in accordance with aspects of the present disclosure;

FIG. 2 illustrates conductivity enhancements of three samples, in accordance with aspects of the present disclosure;

FIG. 3 is a workflow to identify clay content, in accordance with aspects of the present disclosure;

FIG. 4 illustrates machine learning algorithms that may be trained based on data stored in a database and used by a data processing system to determine clay content, in accordance with aspects of the present disclosure;

FIG. 5 is a flow diagram of a method for determining whether porous media contains clay, in accordance with aspects of the present disclosure;

FIG. 6 is a flow diagram of another method for determining a clay content of a porous media sample, in accordance with aspects of the present disclosure; and

FIG. 7 illustrates exemplary components of an EM well logging tool configured to facilitate the techniques described herein, in accordance with aspects of the present disclosure.

### DETAILED DESCRIPTION

One or more specific embodiments of the present disclosure will be described below. These described embodiments are examples of the presently disclosed techniques. Additionally, in an effort to provide a concise description of these embodiments, certain features of an actual implementation may not be described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would still be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the terms “connect,” “connection,” “connected,” “in connection with,” and “connecting” are used to mean “in direct connection with” or “in connection with via one or more elements”; and the term “set” is used to mean “one element” or “more than one element.” Further, the terms “couple,” “coupling,” “coupled,” “coupled together,” and “coupled with” are used to mean “directly coupled together” or “coupled together via one or more elements.” As used herein, the terms “up” and “down,” “uphole” and “downhole”, “upper” and “lower,” “top” and “bottom,” and other like terms indicating relative positions to a given point or element are utilized to more clearly describe some elements. Commonly, these terms relate to a reference point as the surface from which drilling operations are initiated as being the top (e.g., uphole or upper) point and the total depth along the drilling axis being the lowest (e.g., downhole or lower) point, whether the well (e.g., wellbore, borehole) is vertical, horizontal or slanted relative to the surface.

In addition, as used herein, the terms “real time”, “real-time”, or “substantially real time” may be used interchangeably and are intended to described operations (e.g., computing operations) that are performed without any human-perceivable interruption between operations. For example, as used herein, data relating to the systems described herein may be collected, transmitted, and/or used in control computations in “substantially real time” such that data readings, data transfers, and/or data processing steps occur once every second, once every 0.1 second, once every 0.01 second, or even more frequent, during operations of the systems (e.g., while the systems are operating). In addition, as used herein, the terms “automatic” and “automated” are intended to describe operations that are performed are caused to be performed, for example, by a data processing system (i.e., solely by the data processing system, without human intervention).

Oil and gas exploration organizations may make certain oil and gas production decisions, such as determining where to drill, based on well log data. More specifically, a well logging downhole tool may obtain well logging measurements, which may be processed (e.g., normalized, de-noised, provided as inputs to a model, etc.) by a suitable computing device to generate the well log data. As referred to herein, the “well log” is a measurement or a property derived from measurements versus depth or time, or both, of one or more properties (e.g., resistivity, conductivity, dip and azimuth, and so forth) in or around a wellbore and, thus, may be used to identify a location within the wellbore that corresponds to an area of interest (e.g., hydrocarbons, an organic deposit, a “bed” or layer of sedimentary rock, or stratum, and the like). At least in some instances, the well log data may be transformed into one or more visual representations (e.g., a well log) that may be presented as hard copies or on an electronic display, where each visual representation of the one or more visual representations may depict the well log data resulting from the well logging measurements.

One type of well logging measurement that may be used to inform the oil and gas production decisions are electromagnetic (EM) well logging measurements. In general, EM well logging measurements may be obtained using one or more electromagnetic well logging tools that each include a pair of transmitter coils and receiver coils. Conventional EM well logging tools (e.g., EM well logging tools using only coaxial transmitter coils and coaxial receiver coils) may obtain EM well logging measurements (e.g., induction well logging measurements or propagation well logging measurements) that are processed to generate resistivity or conductivity well logs.

As discussed above, accurate determination of a clay-containing formation is crucial in evaluating hydrocarbon reservoirs. The presence of clay impacts reservoir and formation petrophysical properties, and might result in misleading results, when interpreting for properties such as porosity, permeability, and most critical water saturation. There are several defined methods for quantifying the clay content, most acceptable techniques using the surface measurement of x-ray diffraction (XRD). Quantifying the clay content in a reservoir generally requires advanced logging techniques not always available, such as high definition spectroscopy.

The embodiments described herein include a new technique to quantify the clay content downhole in the reservoirs. Electromagnetic conductivity (or resistivity) logs may be initially logged for a required formation, then the formation may be heated and, then, conductivity/resistivity may be logged again. Using a ratio between the two conductivity logs at different temperatures and, in comparison, to baseline results (e.g., on clean sand, clay-free), the clay content may be identified. Clay-containing samples show higher conductivity enhancement at higher temperatures, compared to clay-free sample. The techniques described herein are equally applicable in both laboratory core analysis as well as downhole logging and log analysis.

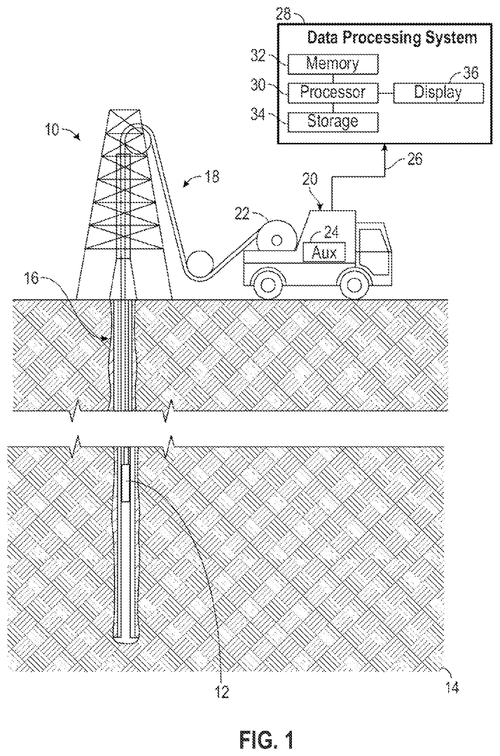

With this in mind, FIG. 1 illustrates a well logging system 10 that may employ the systems and methods of this disclosure. The well logging system 10 may be used to convey an electromagnetic (EM) well logging tool 12 through a geological formation 14 via a wellbore 16. In certain embodiments, the EM well logging tool 12 may be conveyed on a cable 18 via a logging winch system 20. Although the logging winch system 20 is schematically shown in FIG. 1 as a mobile logging winch system carried by a truck, in other embodiments, the logging winch system 20 may be substantially fixed (e.g., a long-term installation that is substantially permanent or modular). Any suitable cable 18 for well logging may be used. The cable 18 may be spooled and unspooled on a drum 22 and an auxiliary power source 24 may provide energy to the logging winch system 20 and/or the EM well logging tool 12.

Moreover, although the EM well logging tool 12 is described herein as a wireline downhole tool, it should be appreciated that any suitable conveyance may be used. For example, the EM well logging tool 12 may instead be conveyed as a logging-while-drilling (LWD) tool as part of a bottom hole assembly (BHA) of a drill string, conveyed on a slickline or via coiled tubing, and so forth. Many types of EM well logging tools 12 may obtain EM logging measurements in the wellbore 16. The EM well logging tool 12 may provide EM logging measurements 26 to a data processing system 28 via any suitable telemetry (e.g., via electrical signals pulsed through the geological formation 14 or via mud pulse telemetry). The data processing system 28 may process the EM logging measurements 26 to identify a horizontal conductivity and/or horizontal resistivity, a vertical conductivity and/or vertical resistivity, a dip and an azimuth at various depths of the geological formation 14 in the wellbore 16.

To this end, the data processing system 28 may be any electronic data processing system that can be used to carry out the systems and methods of this disclosure. For example, the data processing system 28 may include a processor 30, which may execute instructions stored in memory 32 and/or storage 34. As such, the memory 32 and/or the storage 34 of the data processing system 28 may be any suitable article of manufacture that can store the instructions. The memory 32 and/or the storage 34 may be ROM memory, random-access memory (RAM), flash memory, an optical storage medium, or a hard disk drive, to name a few examples. In certain embodiments, a display 36, which may be any suitable electronic display, may provide a visualization, a well log, or other indication of properties in the geological formation 14 or the wellbore 16 using the EM logging measurements 26.

The embodiments described herein provide new and robust methods to identify and quantify clay content in a formation 14 using resistivity logging obtained by the EM well logging tool 12, as described in greater detail herein. These methods rely on using the concept of heating a pay zone or a section of interest in a reservoir formation 14. The conductivity/resistivity of the formation 14 may be logged before and after heating. Using a conductivity ratio between the two measurements (at different temperatures) and, in comparison, to the generated data on clay-free samples, accurate quantification of clay content within the formation 14 may be determined by the data processing system 28.

The embodiments described herein enable quantification of clay content in porous media in general, and in oil and water reservoirs in particular. Using the measured conductivity/resistivity of the formation 14 at two different temperatures, a conductivity enhancement ratio (Cₑᵣ) may be calculated using Eq. 1.

Conductivity Enhancement,Cₑᵣ=Formation Conductivity T₂/Formation Conductivity T₁=C_{T2}/C_{T1} (1)

where T₂ is any higher temperature, at which resistivity is measured, and T₁ is any lower or ambient temperature, at which the resistivity is measured.

Using the calculated conductivity enhancement at different temperatures on samples containing clay, and comparing it to the generated data of clean sand/clay-free samples, the data processing system 28 may identify if the formation 14 has clay or not, and further to quantify the clay content in the formation 14. In clay-containing formations 14, the conductivity enhancement is more pronounced compared to formations 14 with no clay, which will clearly identify and quantify clay content.

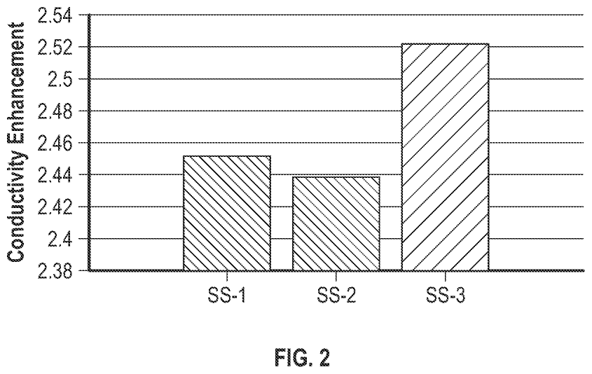

The techniques described herein have been validated with various sets of samples (e.g., samples SS-1, SS-2, and SS-3). These samples have porosity of 20% and permeability of 250 millidarcy (mD). SS-1 and SS-2 are clay-free samples and the SS-3 sample contains illite and muscovite clays of 12.9%. The measured resistivity data is summarized in Table 1 below. The data may then be converted to conductivity and, using Eq. 1, the conductivity enhancements may be calculated for the samples, as summarized in Table 1. For these calculations, the conductivity at 25° C. was considered as the baseline (at T₁). Now, comparing the generated data on sample SS-3 and comparing it with the data of SS-1 and SS-2, a clear difference in the conductivity enhancement is shown in FIG. 2, which confirms the techniques described herein to identify and quantify clay content in a clay-containing formation 24. The SS-1 and SS-2 samples show almost identical conductivity enhancement (e.g., 2.451 and 2.438, respectively, which are approximately 0.5% different from each other), which can then represent a general value for clay-free sandstone formations 14. However, in contrast, the SS-3 sample has a significantly higher conductivity enhancement than the SS-1 and SS-2 samples (e.g., 2.522 versus 2.451 and 2.438, which is approximately 3.0% higher than the other two).

TABLE 1
Measured resistivity and calculated resistivity factor and conductivity
enhancement for samples at elevated temperatures

| | | Resistivity | Conductivity | Conductivity |
|---|---|---|---|---|
| Sample | T [C.] | [ohm · m] | [S/m] | Enhancement |
| SS-1 | 25 | 1.738 | 0.575 | 1.000 |
| | 100 | 0.709 | 1.410 | 2.451 |
| SS-2 | 25 | 1.592 | 0.628 | 1.000 |
| | 100 | 0.653 | 1.531 | 2.438 |
| SS-3 | 25 | 0.991 | 1.009 | 1.000 |
| | 100 | 0.393 | 2.545 | 2.522 |

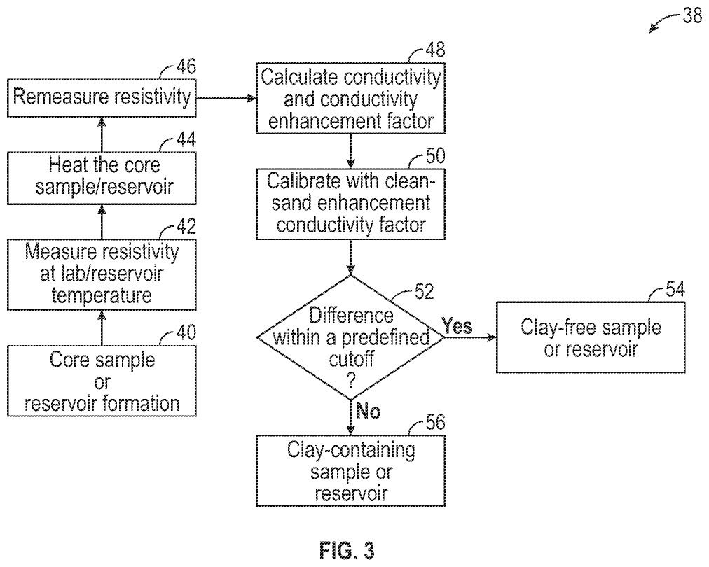

FIG. 3 is a workflow 38 to identify clay content for subsurface applications, as described in greater detail herein. As illustrated, the workflow 38 may begin with either a core sample being collected (e.g., by the EM well logging tool 12 of the well logging system 10 of FIG. 1) for testing at a laboratory (e.g., which is located at the surface of the well logging system 10 or is external to the well logging system 10) or the reservoir formation 14 may be directly sampled downhole within the wellbore 16 of the well logging system 10 by the EM well logging tool 12 (block 40). Then, the resistivity of either the collected sample or the reservoir directly may be measured (e.g., either at the laboratory or by the EM well logging tool 12) (block 42). Then, either the collected sample or the reservoir itself (e.g., proximate the wellbore 16) may be heated (e.g., either at the laboratory or by the EM well logging tool 12) (block 44) and the resistivity of either the collected sample or the reservoir may be remeasured (e.g., either at the laboratory or by the EM well logging tool 12) (block 46).

Once the resistivity of either the collected sample or the reservoir have been determined (e.g., at the two different temperatures), these two separate resistivities may be used by the data processing system 28 to determine a clay content of the collected sample or the reservoir. In particular, first, the conductivity of the collected sample or the reservoir at the two different temperatures may be calculated by the data processing system 28 (e.g., as the inverse of the resistivity of the collected sample or reservoir), which may then be used to calculate the conductivity enhancement ratio (Cₑᵣ) of the collected sample or the reservoir, as described in greater detail herein (block 48). Then, in certain embodiments, the calculated conductivity enhancement ratio (Cₑᵣ) of the collected sample or the reservoir may be calibrated with a clean-sand enhancement conductivity factor by the data processing system 28 (block 50). It will be appreciated that the clean-sand enhancement conductivity factor is a conductivity enhancement ratio (Cₑᵣ) of a relatively clay-free sample.

Then, a determination may be made by the data processing system 28 as to whether a difference between the calculated conductivity enhancement ratio (Cₑᵣ) of the collected sample or the reservoir and the clean-sand enhancement conductivity factor within a predetermined cutoff value (decision block 52). For example, in certain embodiments, as discussed above, in certain embodiments, the predetermined cutoff value may be approximately 1.0%, 0.5%, or even smaller such that the collected sample or the reservoir is considered to be sand-free if the conductivity enhancement ratio (Cₑᵣ) of the collected sample or the reservoir is within 1.0%, 0.5%, or even less than the clean-sand enhancement conductivity factor.

If the difference between the calculated conductivity enhancement ratio (Cₑᵣ) of the collected sample or the reservoir and the clean-sand enhancement conductivity factor is determined by the data processing system 28 to be within the predetermined cutoff value, then the data processing system 28 may determine that the collected sample or the reservoir is relatively clay-free (block 54). Conversely, if the difference between the calculated conductivity enhancement ratio (Cₑᵣ) of the collected sample or the reservoir and the clean-sand enhancement conductivity factor is determined by the data processing system 28 to not be within the predetermined cutoff value, then the data processing system 28 may determine that the collected sample or the reservoir contains a relatively significant amount of clay (block 56).

As such, in certain embodiments, once the formation depth is identified (e.g., by the EM well logging tool 12), then electromagnetic data, such as a resistivity log, may be obtained by the EM well logging tool 12 at a current reservoir temperature. Next, a heating source (e.g., as component of the EM well logging tool 12) may be used to directly heat the formation 14 to a desired temperature, followed by a second resistivity log measurement by the EM well logging tool 12. Next, the formation conductivity may be processed (or the normal output of resistivity from resistivity logging may be converted to conductivity) and the conductivity enhancement ratio (Cₑᵣ) may be calculated by the data processing system 28 using Eq. 1, and compared to a baseline (e.g., clay-free) sample to determine whether the formation 14 contains clay. In other words, in certain embodiments, both direct resistivity measurements as well as direct heating of the reservoir formation 14 may be performed downhole within the wellbore 16 by the EM well logging tool 12. However, in other embodiments, a sample may be collected by the EM well logging tool 12 downhole within the wellbore 16, and the sample may be extracted by the EM well logging tool 12 (e.g., after pulling the EM well logging tool 12 out of the wellbore 16) for testing at a laboratory, at which resistivity measurements of the sample may be measured at two temperatures based on heating of the sample that is done at the laboratory.

In addition to determining whether a collected sample or reservoir contains clay or not, as described in detail with respect to the workflow 38 of FIG. 3, in certain embodiments, the data processing system 28 may be configured to determine a type of clay contained in the collected sample of reservoir. For example, the data processing system 28 may be configured to compare the relative magnitude of the conductivity enhancement ratio (Cₑᵣ) for the collected sample or reservoir to determine that the collected sample or reservoir mostly likely contains a specific type of clay, such as kaolinite, vermiculite, smectite, illite, muscovite, chlorite, and so forth. In particular, it will be appreciated that certain types of clays may have generally higher conductivity enhancement ratios (Cₑᵣ) and the data processing system 28 may be configured to identify that a particular collected sample or reservoir is most likely to contain a particular type of clay based on the magnitude of a conductivity enhancement ratio (Cₑᵣ) for the particular collected sample or reservoir.

In addition to determining whether a collected sample or reservoir contains clay or not, as described in detail with respect to the workflow 38 of FIG. 3, in certain embodiments, the data processing system 28 may be configured to determine a volume of clay contained in the collected sample of reservoir. For example, the data processing system 28 may be configured to compare the relative magnitude of the conductivity enhancement ratio (Cₑᵣ) for the collected sample or reservoir to determine that the collected sample or reservoir mostly likely contains a higher or lower volume of clay that another similar collected sample or reservoir.

In addition, although described primarily herein as determining the existence of clay (and, in certain embodiments, a type and/or volume of the clay) within a collected sample or reservoir by determining resistivity of the collected sample or reservoir at two different temperatures, in other embodiments, the data processing system 28 may be configured to determine a curve of conductivity enhancement ratios (Cₑᵣ) for a collected sample or reservoir at a plurality of temperatures including three or more temperatures. In such embodiments, the shapes of the conductivity enhancement ratio (Cₑᵣ) curves may further enhance the ability of the data processing system 28 to determine the existence of clay (and, in certain embodiments, a type and/or volume of the clay) within a collected sample or reservoir. In particular, it will be appreciated that conductivity enhancement ratio (Cₑᵣ) curves for certain types of clays may have different shapes than others.

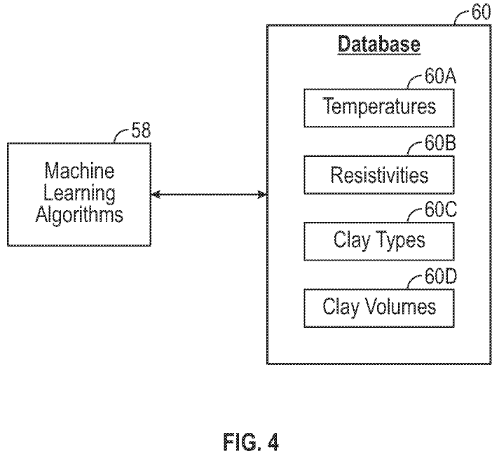

As illustrated in FIG. 4, to enable the data processing system 28 to analyze the conductivity enhancement ratios (Cₑᵣ) and the conductivity enhancement ratio (Cₑᵣ) curves as described in greater detail herein, in certain embodiments, the data processing system 28 may include machine learning algorithms 58 (e.g., stored in the storage 34 of the data processing system 28) that are configured to be trained based on previous data to assist the data processing system 28 to more accurately determine when a particular collected sample or reservoir contains clay (or certain types of clay). In certain embodiments, the machine learning algorithms 58 of the data processing system 28 may be trained based on data collected for previous collected samples or reservoirs, such as temperatures 60A of previous collected samples or reservoirs, resistivities 60B of the previous collected samples or reservoirs, known clay types 60C of the previous collected samples or reservoirs, known clay volumes 60D of the previous collected samples or reservoirs, and so forth, to determine relationships between the data collected for the previous collected samples or reservoirs which may be used to analyze future collected samples or reservoirs. In certain embodiments, the determined relationships (as well as the previous data) may be used to create a database 60 of information that may be used by the data processing system 28 (e.g., and stored in the storage 34 of the data processing system 28, in certain embodiments) to determine the existence of clay (and, in certain embodiments, a type and/or volume of the clay) within collected samples or reservoirs based on resistivities of the collected samples or reservoirs at various temperatures, as described in greater detail herein.

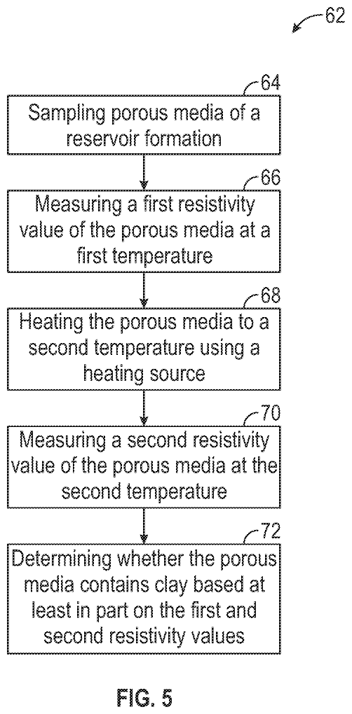

FIG. 5 is a flow diagram of a method 62 for determining whether porous media (e.g., the collected samples or reservoirs described herein) contains clay. In certain embodiments, the method 62 may include sampling porous media of a reservoir formation 14 (block 64). In addition, in certain embodiments, the method 62 may include measuring a first resistivity value of the porous media at a first temperature (block 66). In addition, in certain embodiments, the method 62 may include heating the porous media to a second temperature using a heating source (block 68). In addition, in certain embodiments, the method 62 may include measuring a second resistivity value of the porous media at the second temperature (block 70). In addition, in certain embodiments, the method 62 may include determining whether the porous media contains clay based at least in part on the first and second resistivity values (block 72). In certain embodiments, the porous media includes a core sample collected from the reservoir formation 14, soil collected from the reservoir formation 14, drill cutting samples collected while drilling through the reservoir formation 14, or some combination thereof.

In addition, in certain embodiments, the method 62 may include calculating a conductivity enhancement ratio (Cₑᵣ) based at least in part on the first and second resistivity values; and determining whether the porous media contains clay based at least in part on the conductivity enhancement ratio (Cₑᵣ). In addition, in certain embodiments, calculating the conductivity enhancement ratio (Cₑᵣ) may include converting the first and second resistivity values to first and second conductivity values; and dividing the second conductivity value by the first conductivity value to determine the conductivity enhancement ratio (Cₑᵣ). In addition, in certain embodiments, determining whether the porous contains clay includes comparing the conductivity enhancement ratio (Cₑᵣ) to a clean-sand enhancement conductivity factor.

In addition, in certain embodiments, the method 62 may include determining a clay type in the porous media based at least in part on the first and second resistivity values. In addition, in certain embodiments, the method 62 may include determining a volume of clay in the porous media based at least in part on the first and second resistivity values. In addition, in certain embodiments, the measuring and heating steps of the method 62 may be performed at a laboratory located at a surface location. In addition, in certain embodiments, the measuring and heating steps of the method 62 may be performed by a downhole well tool 12 disposed in a wellbore 16 extending through the reservoir formation 14. In addition, in certain embodiments, the method 62 may include utilizing machine learning algorithms 68 to determine whether the porous media contains clay based at least in part on the first and second resistivity values.

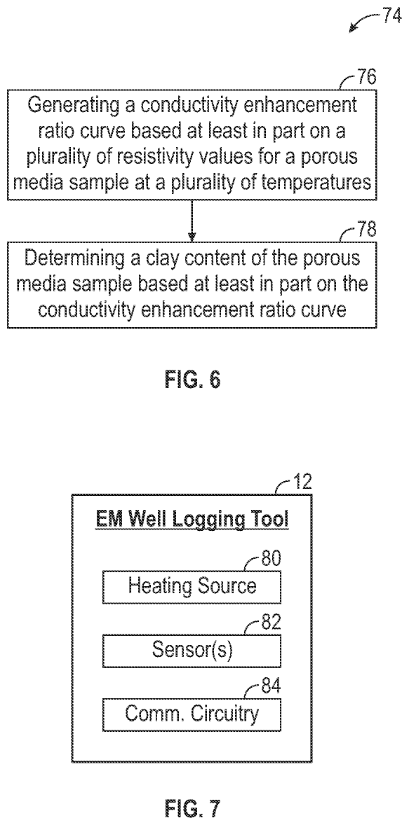

FIG. 6 is a flow diagram of another method 74 for determining a clay content of a porous media sample (e.g., the collected samples or reservoirs described herein). In certain embodiments, the method 74 may include generating a conductivity enhancement ratio (Cₑᵣ) curve based at least in part on a plurality of resistivity values for a porous media sample at a plurality of temperatures (block 76). In addition, in certain embodiments, the method 74 may include determining a clay content of the porous media sample based at least in part on the conductivity enhancement ratio (Cₑᵣ) curve (block 78).

In addition, in certain embodiments, generating the conductivity enhancement ratio curve (Cₑᵣ) may include converting the plurality of resistivity values to a plurality of conductivity values; and comparing the plurality of conductivity values to a clean-sand enhancement conductivity factor. In addition, in certain embodiments, the method 74 may include determining a clay type in the porous media sample based at least in part on the conductivity enhancement ratio (Cₑᵣ) curve. In addition, in certain embodiments, the method 74 may include determining a volume of clay in the porous media sample based at least in part on the conductivity enhancement ratio (Cₑᵣ) curve. In addition, in certain embodiments, the method 74 may include utilizing machine learning algorithms 58 to determine the clay content of the porous media sample based at least in part on the conductivity enhancement ratio (Cₑᵣ) curve.

As described in greater detail herein, the heating of a porous media sample and measurement of resistivity values may be performed either at a surface laboratory or downhole by an EM well logging tool 12. To enable the EM well logging tool 12 to perform these functions, as illustrated in FIG. 7, in certain embodiments, the EM well logging tool 12 may include, among other components, a heating source 80 configured to heat a reservoir formation 14 surrounding a wellbore 16 while the EM well logging tool 12 is disposed within the wellbore 16. In addition, in certain embodiments, the EM well logging tool 12 may include one or more sensors 82 (e.g., EM detection sensors) configured to detect resistivity values of the reservoir formation 14 at a plurality of temperatures while the EM well logging tool 12 is disposed within the wellbore 16. In addition, in certain embodiments, the EM well logging tool 12 may include communication circuitry 84 configured to transmit the resistivity values of the reservoir formation 14 (e.g., to the data processing system 28) for analysis.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112 (f).

1. A method, comprising:
sampling porous media of a reservoir formation;
measuring a first resistivity value of the porous media at a first temperature;
heating the porous media to a second temperature using a heating source;
measuring a second resistivity value of the porous media at the second temperature; and
determining whether the porous media contains clay based at least in part on the first and second resistivity values.

2. The method of claim 1, wherein the porous media comprises a core sample collected from the reservoir formation, soil collected from the reservoir formation, drill cutting samples collected while drilling through the reservoir formation, or some combination thereof.

3. The method of claim 1, comprising:
calculating a conductivity enhancement ratio based at least in part on the first and second resistivity values; and
determining whether the porous media contains clay based at least in part on the conductivity enhancement ratio.

4. The method of claim 3, wherein calculating the conductivity enhancement ratio comprises:
converting the first and second resistivity values to first and second conductivity values; and
dividing the second conductivity value by the first conductivity value to determine the conductivity enhancement ratio.

5. The method of claim 3, wherein determining whether the porous contains clay comprises comparing the conductivity enhancement ratio to a clean-sand enhancement conductivity factor.

6. The method of claim 1, comprising determining a clay type in the porous media based at least in part on the first and second resistivity values.

7. The method of claim 1, comprising determining a volume of clay in the porous media based at least in part on the first and second resistivity values.

8. The method of claim 1, wherein the measuring and heating steps of the method are performed at a laboratory located at a surface location.

9. The method of claim 1, wherein the measuring and heating steps of the method are performed by a downhole well tool disposed in a wellbore extending through the reservoir formation.

10. The method of claim 1, comprising utilizing machine learning algorithms to determine whether the porous media contains clay based at least in part on the first and second resistivity values.

11. A method, comprising:
generating a conductivity enhancement ratio curve based at least in part on a plurality of resistivity values for a porous media sample at a plurality of temperatures; and
determining a clay content of the porous media sample based at least in part on the conductivity enhancement ratio curve.

12. The method of claim 11, wherein generating the conductivity enhancement ratio curve comprises:
converting the plurality of resistivity values to a plurality of conductivity values; and
comparing the plurality of conductivity values to a clean-sand enhancement conductivity factor.

13. The method of claim 11, comprising determining a clay type in the porous media sample based at least in part on the conductivity enhancement ratio curve.

14. The method of claim 11, comprising determining a volume of clay in the porous media sample based at least in part on the conductivity enhancement ratio curve.

15. The method of claim 11, comprising utilizing machine learning algorithms to determine the clay content of the porous media sample based at least in part on the conductivity enhancement ratio curve.

16. A system, comprising:
a downhole well tool comprising:
a heating source configured to heat a reservoir formation surrounding a wellbore while the downhole well tool is disposed within the wellbore;
one or more sensors configured to detect resistivity values of the reservoir formation at a plurality of temperatures while the downhole well tool is disposed within the wellbore; and
communication circuitry configured to transmit the resistivity values of the reservoir formation; and
a data processing system configured to:
receive the resistivity values of the reservoir formation from the downhole well tool;
generate a conductivity enhancement ratio curve based at least in part the resistivity values of the reservoir formation; and
determine a clay content of the porous media sample based at least in part on the conductivity enhancement ratio curve.

17. The system of claim 16, wherein the data processing system is configured to generate the conductivity enhancement ratio curve by:
converting the resistivity values to respective conductivity values; and
comparing the conductivity values to a clean-sand enhancement conductivity factor.

18. The system of claim 16, wherein the data processing system is configured to determine a clay type in the porous media sample based at least in part on the conductivity enhancement ratio curve.

19. The system of claim 16, wherein the data processing system is configured to determine a volume of clay in the porous media sample based at least in part on the conductivity enhancement ratio curve.

20. The system of claim 16, wherein the data processing system is configured to determine utilize machine learning algorithms to determine the clay content of the porous media sample based at least in part on the conductivity enhancement ratio curve.
